(19) United States
(12) Patent Application Publication
(10) Pub. No.: US 2001/0018060 A1
(43) Pub. Date: Aug. 30, 2001

(54) Use of particular fatty substances which make it possible to modify the physicochemical properties of the skin and/or the mucous membranes as agents preventing or reducing the adhesion of microorganisms to the latter

(76) Inventors: Geraldine Lerebour, Athis Mons (FR); Laurence Arnaud-Sebillotte, L'Hay Les Roses (FR)

Correspondence Address: NIXON & VANDERHYE P.C., 8th Floor, 1100 North Glebe Road, Arlington, VA, 22201, US

(21) Appl. No.: 09/782,520

(22) Filed: Feb. 14, 2001

(30) Foreign Application Priority Data
Feb. 15, 2000 (FR) 0001842

(51) Int. Cl. A61K 7/00; A61K 35/78

(52) U.S. Cl. 424/401; 424/757; 424/769; 424/764; 424/735

(57) ABSTRACT

The present invention relates to the use, as active ingredient, in a cosmetic composition or for the preparation of a pharmaceutical composition, of an effective quantity of at least one fatty substance free of carbohydrate units, having a melting point of less than 35° C. and having an interfacial tension of between 6 and 27 mN/m, modifying the physicochemical properties of the surface of the skin and/or of the mucous membranes so as to prevent or reduce the adhesion of microorganisms to the latter.

The invention relates to the use of particular fatty substances which make it possible to modify the physicochemical properties of the surface of the skin and/or the mucous membranes in a cosmetic composition or for the preparation of a pharmaceutical composition as agents preventing or reducing the adhesion of microorganisms, particularly bacteria, to the skin and/or the mucous membranes.

The human skin is permanently populated by a multitude of different microorganisms (bacteria, yeasts and fungi). The resident microbial flora, which is essential for good skin health, consists mainly of staphylococci (Staphylococcus epidermis and Staphylococcus hominis), corynebacteria, propionibacteria which are Gram+ such as Propionibacterium acnes, as well as a fungal flora mainly composed of Pytosporum ovale.

Skin infections are most often due to the disruption of the ecological balance among the resident flora following colonization of the skin by pathogenic exogenous microorganisms or following abnormal proliferation of an endogenous strain. The best known pathogenic microorganisms are Pseudomonas aeruginosa (Gram-) which is responsible for small spots, folliculitis, red blotches and pruritus, Candida albicans which can cause inflammation of the corner of the lips, skin candidiasis, pruritus, folliculitis and aphtha, Staphylococcus aureus which can cause spots, folliculitis, impetigo and furuncles, and Streptococcus of group A responsible for impetigo.

To combat these microorganisms, it is common to use antibiotics or bactericides. The use of these compounds poses, nevertheless, the problem of nonspecificity of action affecting indiscriminately the pathogenic flora and the resident flora, and the problem of the risk of appearance of bacterial resistance, as well as problems of skin tolerance (irritations, allergies and the like).

It is also known to reduce or prevent the colonization of surfaces such as the teeth, the skin and/or the mucous membranes, by pathogenic microorganisms by preventing their attachment to these supports. The compounds used as antiadhesion agents described in the prior art are carbohydrates and derivatives of carbohydrates (WO 96 23 479, EP 380 084, U.S. Pat. No. 5,002,759, U.S. Pat. No. 4,859,656, WO 81 03 175, WO 93 14 773, WO 95 15 149, WO 95 07 084 and WO 95 17 898).

However, most carbohydrates constitute a source of carbon for bacteria and fungi. Their presence in cosmetic compositions consequently promotes microbial proliferation and requires increasing the concentration of preservatives (bactericides or bacteriostats). This disadvantage thus outweighs the benefit of the approach consisting in replacing antiobiotic or bactericidal compounds with compounds reducing microbial adherence.

The applicant has found, surprisingly, that particular fatty substances, free of hydrocarbon units, made it possible to significantly reduce microbial adherence to the skin and/or the mucous membranes and to thus prevent the proliferation of potentially pathogenic microorganisms in the absence of antibiotic, bactericidal or fungicidal agents.

These fatty substances, unlike carbohydrates which bind to the microbial receptors to prevent bindings to the glycolipids of the corneocytes, act on the physicochemical properties of the surface of the skin and/or the mucous membranes, these physicochemical properties involving electrodynamic interactions due to Van der Waals forces, Lewis-type acid-base interactions and electrostatic interactions.

In addition, these fatty substances are not bactericidal. Because of this, they do not cause undesirable side effects on the skin and/or the mucous membranes.

The fatty substances according to the invention, when used as active ingredients, make it possible to reduce or prevent the adhesion of a microorganism whose overall surface charge is negative or positive by increasing respectively the negative or positive charge on the skin, so as to cause repulsion between the skin and/or the mucous membranes and the microorganism.

The fatty substances according to the invention, when used as active ingredients, make it possible, in addition, to reduce or prevent the adhesion of a microorganism by limiting as much as possible the Van der Waals type interactions between the skin and/or the mucous membranes and the microorganisms, by promoting the repulsive interactions of the Lewis acid-base type and by limiting the attractive interactions of the Lewis acid-base type between the microorganism and the skin and/or the mucous membranes.

The subject of the invention is therefore the use, as active ingredient, in a cosmetic composition or for the preparation of a pharmaceutical composition, of an effective quantity of at least one fatty substance free of carbohydrate units, having a melting point of less than 35° C. and having an interfacial tension of between 6 and 27 mN/m, modifying the physicochemical properties of the surface of the skin and/or of the mucous membranes so as to prevent or reduce the adhesion of microorganisms to the latter.

The expression to prevent or to reduce the adhesion of microorganisms should be understood to mean that the fatty substance or the composition containing it may be used both preventively, for its capacity to completely or partially prevent the adhesion of microorganism, and curatively for its capacity to facilitate the detachment of the microorganisms.

Moreover, these fatty substances are such that the decimal logarithm of the mean number of viable bacteria adhering to reconstructed epidermis, after a test consisting in bringing the said epidermis into contact with the test compound for 2 hours at 37° C., is at least 0.3, preferably 0.5 and more preferably 1, less than that obtained by a test carried out with water under the same conditions.

The reconstructed epidermis used in the test indicated above is reconstructed human epidermis, equivalent to human skin, sold by EPISKIN.

This test makes it possible to evaluate the modifications in the physicochemical properties of the surface of the skin and/or of the mucous membranes, involving Van der Waals electrodynamic interactions, Lewis-type acid-base interactions and electrostatic interactions.

The experimental protocol of the test will be defined below.

There is preferably used, as active ingredient in a cosmetic composition or for the preparation of a pharmaceutical composition, an effective quantity of fatty substances free of carbohydrate units, having a melting point of less than 35° C. and having an interfacial tension of between 6 and 27 mN/m, chosen from:

Generally C₈-C₃₀ fatty acid triglycerides such as caprylic/capric acid triglycerides and vegetable oils such as wheatgerm, calendula, castor, olive, avocado, sweet almond, groundnut, jojoba, sesame, apricot stone, sunflower and macadamia oils, and shea butter.

Fatty esters comprising one or more linear or branched chains comprising in particular from 8 to 30 carbon atoms, such as hexyl laurate, decyl oleate, octyl dodecyl neopentanoate, isopropyl myristate, isopropyl isostearate, isopropyl stearate, dioctyl adipate, isononyl isononanoate, tartrate of branched C₁₂-C₁₃ dialcohols, such as the product sold under the name Cosmacol ETI by Enichem, and fatty esters with linear or branched chains containing a glyceryl functional group such as for example octoxyglyceryl palmitate (or 2-ethylhexyl glyceryl ether palmitate), such as the product sold under the name Mexanyl GP by Chimex, octoxyglyceryl behenate (or 2-ethylhexyl glyceryl ether behenate), isopropyl palmitate and di(C₁₂-C₁₃ alkyl) malate such as the product sold under the name Cosmacol FM by the company Enichem.

Sesame oil, octoxyglyceryl palmitate, octoxyglyceryl behenate, dioctyl adipate, apricot stone oil, tartrate of branched C₁₂-C₁₃ dialcohols and/or mixtures thereof will be used more preferably still as active ingredient according to the invention.

According to the invention, the fatty substance or the composition containing it is used for topical application to the skin and/or the mucous membranes.

The adhesion of microorganisms to the skin and/or the mucous membranes has consequences which range from mere unpleasantness (odour, small spots and the like) to more serious or less serious diseases.

One of the aspects of the invention is therefore to propose the use of a fatty substance free of carbohydrate units, having a melting point of less than 35° C. and having an interfacial tension of between 6 and 27 mN/m as active ingredient in a cosmetic composition or for the preparation of a pharmaceutical composition.

In particular, the subject of the invention is the cosmetic use by topical application of at least one fatty substance as defined above, as active ingredient in a cosmetic composition intended to reduce bad body odours and/or intended for body hygiene health care.

The expression body hygiene health care is understood to mean any substance or preparation intended to be brought into contact with various superficial parts of the human body and/or with the teeth and/or the mucous membranes so as to clean them, protect them, maintain them in good condition, modify the appearance thereof, perfume them and correct the odour thereof.

In particular, the subject of the invention is the cosmetic use by topical application of at least one fatty substance free of carbohydrate units, having a melting point of less than 35° C. and having an interfacial tension of between 6 and 27 mN/m, as active ingredient in a cosmetic composition intended to combat comedones and/or dandruff.

The microbial flora of the surface of the skin is responsible for a large number of disorders.

Thus, the subject of the invention is also the use of a fatty substance free of carbohydrate units, having a melting point of less than 35° C. and having an interfacial tension of between 6 and 27 mN/m, as active ingredient for the preparation of a pharmaceutical composition intended to be used by topical application to combat mycosis and/or acne, particularly juvenile acne.

The quantity of fatty substance which can be used according to the invention quite obviously depends on the desired effect and should be a quantity effective for partially or completely preventing adhesion of microorganisms or for facilitating the detachment of microorganisms.

By way of example, the quantity of fatty substance used according to the invention may range, for example, from 0.1 to 100%, preferably from 0.5 to 50% and better still from 5 to 25% of the total weight of the composition.

The subject of the invention is also a cosmetic method for treating disorders linked to the adhesion of microorganisms consisting in applying to the skin a cosmetic composition comprising at least one fatty substance according to the invention in a cosmetically acceptable medium.

The expression cosmetically acceptable medium is understood to mean a medium compatible with the skin, the scalp, the mucous membranes, the nails and the hair.

The cosmetic and pharmaceutical compositions used according to the invention may be provided in all the galenic forms normally used for topical application, in particular in the form of liquid, pasty or solid anhydrous products, such as oily lotions, oily gels, unguents, or in the form of oil-in-water or water-in-oil or multiple emulsions, or a dispersion of oil in an aqueous phase with the aid of spherules, it being possible for these spherules to be polymeric nanoparticles such as nanospheres and nanocapsules, or even better, lipid vesicles of ionic and/or nonionic type.

For the compositions of the invention to be more pleasant to use, smoother on application, more nourishing and more emollient, it is possible to add an additional fatty phase to the medium for these compositions while ensuring the anti-adherent efficacy of the mixture.

The additional fatty phase preferably represents from 0 to 50% of the total weight of the composition.

This additional fatty phase may comprise one or more oils preferably chosen from the group consisting of:

volatile or nonvolatile silicones which are linear, branched or cyclic, organomodified or otherwise, water-soluble or fat-soluble,

mineral oils such as paraffin oil and liquid petroleum jelly,

oils of animal origin such as perhydrosqualene,

synthetic oils such as isoparaffins,

fluorinated and perfluorinated oils,

fatty acid esters.

They may also comprise, as additional fatty substances, one or more fatty alcohols, fatty acids or waxes (paraffin, polyethylene wax, Carnauba wax, beeswax).

In a known manner, the compositions used in the invention may, in addition, contain customary adjuvants in the cosmetic field such as solvents; gelling agents and/or hydrophilic or lipophilic conventional thickening agents; hydrophilic or lipophilic active agents; preservatives, antioxidants; perfumes; emulsifiers; moisturizing agents; pigmenting agents; depigmenting agents; keratolytic agents; vitamins; emollients; sequestrants; surfactants; polymers; alkalinizing or acidifying agents; fillers; anti-free radical agents; ceramides; sun screens (in particular ultraviolet-screening agents); insect repellents; slimming agents; colouring matter; anti-dandruff agents.

As surfactants, there may be mentioned, for example, the silicone surfactants such as polydimethicone copolyols and alkyldimethicone copolyols such as for example the product sold under the name Abil EM90 by Goldschmidt; esters of fatty acids and polyols such as PEG 7 glyceryl cocoate such as the product sold by COGNIS under the name Cetiol HE.

As solvents, there may be mentioned hydrophilic organic solvents, and for example linear or branched lower monoalcohols having from 1 to 8 carbon atoms such as ethanol, propanol, butanol, isopropanol, isobutanol; polyethylene glycols having from 6 to 80 ethylene oxides, polyols such as propylene glycol, isoprene glycol, butylene glycol, glycerol; mono- or dialkyls of isosorbide in which their alkyl groups have from 1 to 5 carbon atoms such as dimethyl isosorbide; glycol ethers such as diethylene glycol monomethyl or monoethyl ether and ethers of propylene glycol such as dipropylene glycol methyl ether.

The quantities of these various adjuvants are those conventionally used in the fields considered.

Of course, persons skilled in the art will be careful to choose the possible compound(s) to be added to the composition according to the invention such that the advantageous properties intrinsically attached to the composition in accordance with the invention are not, or not substantially, adversely modified by the addition envisaged.

The compositions used in the present invention may be fluid to a greater or lesser degree and may have the appearance of a white or coloured cream, an ointment, a milk, a lotion, a serum, a paste, a foam or a solid.

They may be optionally applied to the skin in aerosol form.

They may be provided in solid form, and for example in the form of a stick.

They may be used as health care product, as cleansing product for the skin or the hair, as sun screen product, as make-up product such as foundations, lipsticks, mascaras, blushers, and/or as simple deodorant product.

Thus, the subject of the invention is a cosmetic health care, cleansing, make-up or deodorant composition comprising at least one fatty substance according to the invention.

The antiadhesion test corresponds to the protocol below:

Before bacterial adhesion, the reconstructed epidermis is brought into contact for 2 hours with 25 mg of the fatty substance to be tested at 37° C. 1 ml of bacterial suspension of Staphylococcus aureus at a concentration of 10⁷ microorganisms/ml in Tryptone salt is then added thereto. After incubating for 24 hours at 37° C., the bacterial suspension is emptied and five rinsings are carried out with 1 ml of sterile distilled water. The epidermis, detached from its support, is then ground with the aid of a food processor in 18 ml of Tryptone salt. A decimal dilution is carried out on this suspension in Tryptone salt, and 1 ml of the dilution is then inoculated into 15 ml of Trypticase Soy agar and the medium is incubated for 24 hours at 37° C. The adherent and viable cells are then counted.

This antiadhesion test makes it possible to evaluate the efficacy of molecules alone or of finished products.

Before the antiadhesion test, the following viability test is carried out:

A bacteria/test product mixture, in the same ratio as in the antiadhesion test is brought into contact for 24 hours at 37° C. The test may require incubation, with stirring, in order to avoid the death of the bacteria through lack of oxygen, for certain fatty substances. The microorganisms are counted by decimal dilution in Tryptone salt and inoculated with a 100 μl scraper on Trypticase Soy agar. The colonies are counted after 24 hours of incubation at 37° C.

The test for viability carried out prior to the antiadhesion test makes it possible to rule out any bactericidal component for the molecules or the finished products tested and to demonstrate only the antiadhesion activity.

The following examples present the results obtained for various fatty substances having an interfacial tension of between 6 and 27 mN/m and a melting point of less than 35° C. according to the invention and a particular embodiment of a composition according to the invention.

These examples are of course given by way of illustration and have absolutely no limitative character.

### Examples of fatty substances used according to the invention:

The results obtained for the fatty substances presented here result from the use of the protocol detailed above.

The figures presented opposite the fatty substance correspond to the reduction of the decimal logarithm of the mean number of viable Staphylococcus aureus adhering to reconstructed epidermis after treatment with the fatty substance under the conditions defined by the preceding test compared with the decimal logarithm of the mean number of viable Staphylococcus aureus adhering to reconstructed epidermis after treatment with water under the same conditions.

| | Reduction of |
|---|---|
| Oil | log/control |
| Olive oil | 3.25 |
| Sweet almond oil | 1.34 |
| Sesame oil | 1.92 |
| Apricot stone oil | 0.81 |
| Sunflower oil | 1.4 |
| Dioctyl adipate | 0.9 |
| Tartrate of branched C₁₂-C₁₃ dialcohols | 2.31 |
| Octoxyglyceryl palmitate | 1.85 |
| Octoxyglyceryl behenate | 2.59 |
| Isopropyl palmitate | 1.07 |
| Di(C₁₂₋₁₃ alkyl) malate | 3.68 |

### Example of the importance of the use of fatty substances having an interfacial tension of between 6 and 27 mN/m:

The figures presented opposite the fatty substance correspond to the value of the decimal logarithm of the mean number of viable Staphylococcus aureus adhering to reconstructed epidermis after treatment with the fatty substance under the conditions defined by the preceding test compared with the decimal logarithm of the mean number of viable Staphylococcus aureus adhering to reconstructed epidermis after treatment with water under the same conditions.

| | Interfacial | |
|---|---|---|
| Oil | tension | Log measured |
| Squalane | 46 mN/m | Increase of |
| | | 0.27/control |
| Hydrogenated | 40 mN/m | Without effect |
| polyisobutene | | |

### Example of composition used according to the invention:

W/O emulsion for face care:

| | |
|---|---|
| Oxyethylenated polymethylcetyl dimethyl | 3% |
| methylsiloxane (ABIL EM 90 ® from | |
| GOLDSCHMIDT) | |
| Palmitate of 2-ethylhexyl glyceryl ether | 10% |
| (MEXANYL GP ® from CHIMEX) | |
| Tartrate of branched C₁₂-C₁₃ dialcohols | 10% |
| (COSMACOL ETI ® from ENICHEM) | |
| Oxyethylenated glyceryl cocoate (7 EO) | 3% |
| (CETIOL HE ® from GOGNIS) | |
| Condensate of ethylene oxide, propylene | 3% |
| oxide and ethylene oxide (MW: 8 350) | |
| (75 EO/30 PO/75 EO) (LUTROL F 68 ® from | |
| BASF) | |
| Water QS | 100 |
| Antioxidant | qs |
| Perfume | qs |

After treatment with the above composition, under the conditions defined by the preceding test, a decrease of 3.96 in the decimal logarithm of the mean number of viable Staphylococcus aureus adhering to reconstructed epidermis is observed compared with the decimal logarithm of the mean number of viable Staphylococcus aureus adhering to the reconstructed epidermis after treatment with water under the same conditions.

1. Use, as active ingredient, in a cosmetic composition or for the preparation of a pharmaceutical composition, of an effective quantity of at least one fatty substance free of carbohydrate units, having a melting point of less than 35° C. and having an interfacial tension of between 6 and 27 mN/m, modifying the physicochemical properties of the surface of the skin and/or of the mucous membranes so as to prevent or reduce the adhesion of microorganisms to the latter.

2. Use according to claim 1, characterized in that the active ingredient is chosen from fatty acid triglycerides, vegetable oils and fatty esters comprising one or more linear or branched chains, comprising 8 to 30 carbon atoms.

3. Use according to either of claims 1 and 2, characterized in that the active ingredient is chosen from caprylic/capric acid triglycerides, wheatgerm oil, calendula oil, castor oil, olive oil, avocado oil, sweet almond oil, groundnut oil, jojoba oil, sesame oil, apricot oil, sunflower oil, macadamia oil, shea butter, hexyl laurate, decyl oleate, octyl dodecyl neopentanoate, isopropyl myristate, isopropyl isostearate, isopropyl stearate, dioctyl adipate, isononyl isononanoate, tartrate of branched C₁₂-C₁₃ dialcohols, octoxyglyceryl palmitate, octoxyglyceryl behenate and/or mixtures thereof, isopropyl palmitate and di(C₁₂-C₁₃ alkyl) malate such as the product sold under the name Cosmacol FM by the company Enichem.

4. Use according to any one of claims 1 to 3, characterized in that the active ingredient is chosen from sesame oil, shea butter, octoxyglyceryl palmitate, octoxyglyceryl behenate, dioctyl adipate, apricot stone oil, tartrate of branched C₁₂-C₁₃ dialcohols and/or mixtures thereof, isopropyl palmitate and di(C₁₂-C₁₃ alkyl) malate such as the product sold under the name Cosmacol FM by Enichem.

5. Use according to any one of claims 1 to 4, characterized in that the active ingredient is present in a quantity ranging from 0.1% to 100% of the total weight of the composition.

6. Use according to claim 5, characterized in that the active ingredient is present in a quantity ranging from 0.5% to 50% of the total weight of the composition.

7. Use according to either of claims 5 and 6, characterized in that the active ingredient is present in a quantity ranging from 5% to 25% of the total weight of the composition.

8. Use according to any one of claims 1 to 7, characterized in that the composition is provided in the form of a lotion, a gel, a serum, an emulsion or a dispersion of lipid vesicles.

9. Method of cosmetic treatment for treating disorders linked to the adhesion of microorganisms consisting in applying to the skin and/or the mucous membranes a cosmetic composition comprising at least one fatty substance according to any one of claims 1 to 7 in a cosmetically acceptable medium.

10. Cosmetic use by topical application of at least one fatty substance free of carbohydrate units, having a melting point of less than 35° C. and having an interfacial tension of between 6 and 27 mN/m as active ingredient in a cosmetic composition intended to reduce bad body odours and/or intended for body hygiene health care.

11. Cosmetic use by topical application of at least one fatty substance free of carbohydrate units, having a melting point of less than 35° C. and having an interfacial tension of between 6 and 27 mN/m as active ingredient in a cosmetic composition intended to combat comedones and/or dandruff.

12. Use of at least one fatty substance free of carbohydrate units, having a melting point of less than 35° C. and having an interfacial tension of between 6 and 27 mN/m as active ingredient for the preparation of a pharmaceutical composition intended to be used by topical application to combat mycosis and/or acne.
